(19) United States
(12) Patent Application Publication
(10) Pub. No.: US 2021/0170415 A1
(43) Pub. Date: Jun. 10, 2021

(54) LABORATORY TEMPERATURE CONTROL DEVICES

(71) Applicant: Eppendorf AG, Hamburg (DE)

(72) Inventors: Jan FITZER, Hamburg (DE); Philipp ABEL, Hamburg (DE); Sören MENSCH, Hamburg (DE); Lutz TIMMANN, Hamburg (DE)

(21) Appl. No.: 17/046,230

(22) PCT Filed: Apr. 9, 2019
(86) PCT No.: PCT/EP2019/058985

§ 371 Date: Dec. 22, 2020

(30) Foreign Application Priority Data
Apr. 9, 2018 (EP) 18166337.8

(51) Int. Cl. B01L 7/00; C12M 1/00

(52) U.S. Cl. CPC B01L 7/00; C12M 41/14; B01L 2300/0663; B01L 2200/0689; B01L 2300/027; B01L 2200/147

(57) ABSTRACT

The invention relates to laboratory temperature control devices for storing laboratory samples. It particularly concerns incubators for the growth of cell cultures. Efficient measures for thermal decoupling of chamber and housing of the laboratory temperature control device are described.

The invention concerns a laboratory temperature control device for storing laboratory samples at a target temperature. It concerns in particular an incubator for the growth of cell cultures.

Laboratory temperature control devices are required to store laboratory samples in a shielded environment at a specific target temperature. Incubators are used in biological and medical laboratories to keep cells in cell culture under controlled environmental conditions to enable the growth of living cells in vitro. For this purpose, the temperature and the gas composition or the humidity of the atmosphere inside an incubator chamber isolated from the environment are kept at the desired values by the equipment of the incubator. Eukaryotic cells must be cultivated in CO₂ incubators. The atmosphere is formed by air with a certain CO₂ and O₂ content and a certain humidity. A suitable temperature is often 37° C., although these parameters are usually adjustable. In order to reliably guarantee the environmental conditions required for each cell, a homogeneous temperature distribution or a homogeneous climate in the incubator chamber as well as an insensitivity to external influences is desirable.

Such laboratory temperature control devices have a chamber for holding the laboratory samples to be temperature controlled. This chamber is usually located inside a housing and is separated from it at least in sections by insulating material. Access to the chamber, where the user stores and retrieves the samples inside the housing, especially inside the chamber, is usually via a chamber opening or housing opening that can be closed by a housing door. A problem of state-of-the-art laboratory temperature control devices is that thermal bridges form at the connection points between the housing and the chamber, which may be located particularly in the area of the chamber opening or housing opening, which can lead to undesired disturbances of the chamber climate. With incubators it was observed that condensate forms on the inner chamber walls near the thermal bridges, because at these points heat is locally removed via the thermal bridges leading to the outside, which leads to a local cooling of the inner walls near the joints and to condensation. It is important to avoid the formation of condensate, as it contaminates the interior and serves as a basis for germs to grow. In addition, the thermal bridges lead to a continuous loss of energy. In principle, however, a low energy consumption of the laboratory temperature control devices should be aimed for. Similar properties, i.e. homogeneous temperature distribution, insensitivity to interference and low energy consumption, are also desired for laboratory temperature control devices designed as cooling devices.

It is therefore an object of the present invention to provide an improved laboratory temperature control device whose chamber interior is efficiently thermally decoupled from the environment.

The invention solves this problem by the laboratory temperature control device according to claim 1 and by the laboratory temperature control device according to claim 10. Further technical solutions and preferred configurations are mentioned in the description, and further preferred configurations are also subject of the dependent claims.
The laboratory temperature control device according to the invention, which is in particular an incubator for cell cultures, is used for temperature-controlled storage of laboratory samples and comprises: a housing with a housing interior surrounded by at least one housing wall, a temperature-controlled chamber arranged in the housing with a chamber interior surrounded by at least one chamber wall for receiving the laboratory samples, a plurality of spacer elements, a spacer element having at least a first connecting section, by means of which the spacer element and the housing are connected and has at least one second connecting section spaced from the first connecting section, by means of which the spacer element and the chamber are connected so that the chamber is held spaced from the housing by means of the spacer elements, wherein the spacer elements are each formed using a material with a thermal conductivity of less than 15 W/(mK).

Due to the low thermal conductivity of the spacer elements, they act as thermal insulators. The heat flow between chamber and housing is thus significantly reduced compared to conventional solutions in which chamber and housing are connected by metallic, i.e. excellently conductive, connecting means. The formation of thermal bridges between chamber and housing is thus reduced to a minimum. Since the chamber is held to the housing by spacer elements, there is no need for further connecting elements or flat connecting sections between the chamber and housing, which would form undesirable thermal bridges. On the other hand, the mechanical positional stability of the chamber in the housing is ensured by the spacer elements, whose material and in particular their shape and number can be or are optimized for this task. The provision of several spacer elements makes it possible to reduce the heat-conducting cross-section of the connection between chamber and housing and on the other hand creates the possibility of optimally distributing the mechanical load between chamber and housing. Furthermore, by selecting at least one spacer element, which is designed for floating mounting of the spacer element on the chamber and/or on the housing, it is possible to enable relative movements between chamber and housing in order to prevent thermally induced mechanical stresses.

By using the spacer elements with poor heat conduction properties, it is achieved in particular that in the case of a laboratory temperature control device designed as a CO₂ incubator, condensation of water vapour inside the chamber is prevented at those points of the chamber wall which are connected to the housing with the aid of the spacer elements and in which heat dissipation would lead to a local heat sink and thus to local condensation spots.

Especially preferred is a spacer element formed using a non-metallic material. Such non-metallic materials have a significantly lower thermal conductivity than metals. The thermal conductivity of the material, especially the non-metallic material, is preferably less than 10 W/(mK), preferably less than 5 W/(mK), especially preferably less than 2.5 W/(mK).

Particularly preferred is a spacer element formed using a material comprising plastic, and consists in particular of plastic. The plastic can be reinforced with fibers or fillers. The spacer element can be made of a composite material. Plastic-based materials have a significantly lower thermal conductivity than metals. The thermal conductivity of the material, in particular the plastic-containing material, is preferably less than 2 W/(mK), preferably less than 1 W/(mK), in particular preferably less than 0.8 W/(mK). Particularly preferred is the material a high-performance plastic that is particularly resistant to high temperatures and can tolerate operating temperatures of 180° C. to 200° C. in particular. Such temperatures are used in incubators during sterilization cycles to sterilize the chamber interior. In addition, high-performance plastics can also withstand very low temperatures in laboratory temperature control devices designed as refrigerators and freezers. The materials polyphenylene sulfide (abbre-viation PPS), polyether ether ketone (PEEK), polyether ketone (PEK) and filled polybutylene terephthalate (PBT) have proven to be particularly suitable. Their mechanical properties can be further improved in particular by fiber additives and fillers.

The thermal conductivity of solids is basically a temperature dependent parameter. In the context of the description of the invention, the specification of the thermal conductivity refers by default to a measurement at 20° C. Thermal conductivities of thermally insulating materials can be determined in particular by using the industrial standard DIN 52612-1.

Preferably the spacer element consists mainly of the material with the thermal conductivity lower than 15 W/(mK). Preferably, the spacer element consists entirely, or substantially entirely, of the material with a thermal conductivity of less than 15 W/(mK).

Preferably, the spacer element at least between the first connecting section and the second connecting section is, at least in sections, in particular in a third section, made of the material with a thermal conductivity of less than 15 W/(mK). This ensures that the heat flow in the spacer element is completely or substantially complete over a section, especially the third section, which has a thermal conductivity of less than 15 W/(mK).

The thermal decoupling between the first and the second connecting section depends not only on the material property “thermal conductivity”, but also on the geometrical condition of the spacer element. For an ideal straight heat conductor or thermal insulator with the cross-sectional area A and the length L and the thermal conductivity λ, the change of heat over time, i.e. the heat flow \dot{Q} caused by a temperature difference ΔT is given by

$$\dot{Q} = λ \cdot \frac{A}{L} \cdot Δ T .$$

The longer the distance between the first and second connection section over which the heat flow occurs, the smaller the heat flow and the better the thermal decoupling; the shorter this distance, the worse the thermal decoupling. The smaller the cross-section of the spacer element transverse to the direction of the heat flow between the first and second connection section, the smaller the heat flow and the better the thermal decoupling, the larger this cross-section, the worse the thermal decoupling. Due to the demands on the mechanical load-bearing capacity of the spacer element, however, it cannot be made as thin and long as desired. The solution according to a preferred embodiment of the invention is that the spacer element—with a given size and shape—comprises at least one recess and/or at least one cavity.

Such a cavity or recess makes it possible to provide a spacer element which, on the one hand, generates a low heat flow between the first and the second connecting section and, on the other hand, has sufficient mechanical strength.

A first connecting section is considered to be such an area of the spacer element which, in the connecting position in which the chamber and the housing are connected by the spacer element, is arranged on the housing and in particular contacts the latter. Preferably, the first connection section is mounted on the housing, in particular mounted slidingly. A second connecting section is considered to be that area of the spacer element which is located at the chamber in the connecting position and contacts it in particular. Preferably, the second connecting section is mounted on the chamber, and can also have a sliding bearing.

Preferably, a spacer element comprises at least one cavity and/or at least one recess. Preferably, the spacer element comprises at least one section, called in particular the third section, which connects the first and the second connecting section. This at least one third section is preferably web-shaped, which reduces the heat flow and increases the thermal resistance. Along the longitudinal direction of the web, it can be subjected to mechanical load in tension or shear. Preferably, the web-shaped section between the first and second connecting sections is linear, especially along a virtual axis connecting the first and second connecting sections. This means that the mechanical load capacity (pull and/or push) in axial direction is high. Preferably, several third sections are provided, which connect a first and a second connecting section.

Preferably the first and/or the second connecting section of the spacer element comprises at least one hole by means of which the spacer element can be connected to the housing or the chamber. In particular, there may be at least one hole in the form of an oblong hole, which may be configured for sliding support of a connecting element connected to the housing or chamber. Preferably a metal pin, in particular a screw, is provided, which extends through the hole or slot and connects the first or second connecting section to the housing or chamber.

Preferably the spacer element comprises more than one first and/or second connection section. This allows the mechanical forces of the chamber attachment to be distributed even more favorably.

Preferably the spacer element has a plate-shaped section or is plate-shaped. A plate-shaped spacer element has good mechanical strength, especially in directions parallel to the main plane of the plate, and exhibits increased thermal resistance in such directions due to its small cross-section, which is desirable for thermal decoupling. In addition, a plate-shaped component can be advantageously mounted on a parallel surface, especially on a chamber wall or housing wall, which then stabilizes the position of the plate-shaped component.

Preferably, the spacer element has several, in particular interconnected web sections, which in particular are at least partially aligned in the direction of a connecting section at which the chamber is connected to the spacer element.

Preferably the spacer element comprises one or more recesses, openings or cavities and/or is porous at least in sections.

Preferably, the laboratory temperature control device comprises at least one spacer element which is connected to a front wall of the housing and a front wall of the chamber. Preferably, the laboratory temperature control device comprises several spacer elements which are connected to the housing, in particular to a front wall of the housing, and the chamber, in particular to a front wall of the chamber, in the lower area of the chamber. The lower area is in particular a bottom-side front wall of the chamber, which is connected to a bottom-side front wall of the housing by means of the spacer elements. “Bottom-side” means “near the bottom wall”, the bottom wall is a lower outer wall of the chamber or enclosure.

Preferably, the laboratory temperature control device has several spacer elements which are connected to the housing, in particular to a front wall of the housing, and to the chamber, in particular to a front wall of the chamber, in the upper area of the chamber. The upper area is in particular a front wall of the chamber on the ceiling side, which is connected to a front wall of the housing on the ceiling side by means of the spacer elements. “Ceiling side” means “near the ceiling wall”, the ceiling wall is an upper outer wall of the chamber or enclosure.

Preferably at least one spacer element is provided, which is configured for floating mounting of the spacer element on the chamber and/or on the housing. The floating mounting allows a relative movement between chamber and housing, which can be used to prevent thermally induced mechanical stresses in the laboratory temperature control device. The floating mount is achieved in particular by a sliding support of the corresponding first and/or second connecting section of the spacer element. The slide mounting succeeds in particular in that the corresponding connecting section comprises a slotted hole in which a sliding element of the chamber or housing slides and simultaneously effects the connection.

According to a second particular aspect of the invention, a laboratory temperature control device is embodied as follows:
A laboratory temperature control device according to the invention, in particular an incubator for cell cultures comprises:
a housing with an outer door and a housing opening which can be closed by the outer door and which is formed in a housing front wall,
a chamber arranged in the housing and surrounding a chamber interior with a chamber opening which is formed in a chamber front wall,
wherein the housing front wall and the chamber front wall are separated from one another by a first seal which surrounds the chamber opening,
the outer door having an inner side with a second seal which, in the closed state of the outer door, abuts against the first seal and surrounds the chamber opening.

Such a laboratory temperature control device according to the invention offers the advantage that the space formed between the chamber door and the housing door when the outer door is closed is not in contact with the housing. This is due to the fact that, when the outer door is closed, the second seal laterally limits the space between the two seals by ensuring uninterrupted contact with the first seal. Any contact between the air mixture heated by the chamber and the non-tempered outer wall of the housing and thus any convection-induced heat transfer between the interstitial space and the housing is therefore impossible.

The first seal and the second seal are preferably made of an elastomeric material that is resistant to high temperatures, especially up to 200° C. The elastomeric material is in particular an elastomeric plastic, in particular a silicone plastic, in particular a plastic foam, preferably a silicone foam.

The first seal connects the housing front wall and the chamber front wall. The first seal comprises a minimum or average material thickness which, measured perpendicular to the front wall of the housing, is less than this shortest connection or the gap width between the housing front wall and the chamber front wall. In particular, the minimum or average material thickness d2 of the first seal is less than 1 cm, in particular 0.8 cm, preferably between 0.2 cm and 1 cm. The shortest connection is given in particular by the width of the gap between the housing front wall and the chamber front wall. The width of the gap d1 is preferably more than 1.0 cm, and is preferably in the range of more than 1.2 cm, preferably in the range of 1.0 cm to 2.0 cm, preferably between 1.2 cm and 1.8 cm, preferably between 1.2 and 1.6 cm. The width of the gap can also be wider, therefore also the first seal can be wider. Due to the mentioned preferred embodiments (maximum heat flow distance in the direction of the width of the gap and minimum heat conducting cross-section perpendicular to this direction) the thermal resistance of the first seal is maximum.

The second seal preferably comprises a circumferential first sealing area located closer to the outer door, which has a higher modulus of elasticity than a second sealing area, preferably integrally connected to the first sealing area, which is in particular softer and which, in the closed position of the outer door, is closer to the first seal and contacts the latter. A softer sealing area between the outer door and the housing makes it easier to seal the space between the outer door and the chamber door and also reduces the forces required to close the chamber door, making it easier for the user to operate. The first sealing area is preferably manufactured using an elastomer, in particular silicone, the second sealing area is preferably manufactured using a foamed elastomer, in particular silicone foam.

According to a third particular aspect of the invention, a laboratory temperature control device is configured as follows:
A laboratory temperature control device according to the invention, in particular an incubator for cell cultures comprises:
a housing with an outer door and a housing opening which can be closed by the outer door and which is formed in a housing front wall,
a chamber arranged in the housing and surrounding a chamber interior with a chamber opening which is formed in a chamber front wall,
wherein the housing front wall and the chamber front wall are separated by a gap, and
wherein a wall end section of the housing front wall surrounding the chamber front wall forms the housing opening, and wherein the chamber front wall forms a flange around the chamber opening which terminates in a wall end section surrounding the chamber opening,
wherein the wall end section of the housing front wall and the wall end portion of the chamber front wall each have a thickness between 0.5 mm and 4.0 mm, preferably between 0.8 mm and 3.0 mm, and face each other to form said gap.

This configuration minimizes the heat transfer between the wall end section of the housing front wall and the wall end section of the chamber front wall by means of thermal radiation and further improves the thermal decoupling between chamber and housing.

In particular, the wall end section of the housing front wall and the wall end section of the chamber front wall are arranged without overlap with respect to a projection direction perpendicular to the plane of the chamber opening, i.e. the projections of the wall end sections onto this plane do not intersect each other. The in particular planar wall end section of the housing front wall and the in particular planar wall end section of the chamber front wall preferably lie in the same plane.

The laboratory temperature control device for storing laboratory samples is in particular a temperature control cabinet for temperature control of laboratory samples. Such devices are electrically operated and comprise a voltage connection.

The temperature control cabinet regulates the temperature of the laboratory samples, i.e. it keeps the inside of the housing and thus the laboratory samples stored there within the scope of tolerances by temperature control at a setpoint temperature that can be set by the user in particular. This can be above room temperature (ambient temperature), as this is the case with a warming cabinet or incubator, or below room temperature, as this is the case with a refrigerator or freezer. In the case of a laboratory cabinet configured as a climatic cabinet, preferably also a climate parameter prevailing inside the cabinet is controlled within tolerances. This climate parameter can be the air humidity, and/or a gas concentration, e.g. a CO₂, O₂ and/or N₂ concentration. Such a climate cabinet is, for example, an incubator for laboratory samples consisting of living cell cultures.

The laboratory temperature control device preferably comprises a housing. The housing is preferably an external housing whose housing walls are in contact with the environment. The housing door can be accordingly an outer housing door, which in the locking position borders on the environment.

The housing door comprises in particular a hinge mechanism, which connects the housing door pivotably with the housing. Such a swing door is moved by a rotation between an open position and the closing position. In particular, the hinge device can be located at the vertically oriented outer edge of a cuboid-shaped housing, which is adjacent to the housing opening, when the laboratory cabinet device is used as intended. The base plate of a cuboid-shaped housing is arranged horizontally when the laboratory cabinet device is used as intended, the side walls of the housing are arranged in particular vertically, and the top plate of the housing is arranged in particular horizontally opposite to the base plate.

The chamber door or housing door can also be a sliding door, which is moved by a translatory movement between an open position and the closing position. A mixed swivel/translatory movement of the chamber door or housing door is also possible.

A data processing device is preferably part of the electrical control unit, which controls functions of the laboratory temperature control device. The functions of the control unit are implemented in particular by electronic circuits. The control unit may comprise a computing unit (CPU) for the processing of data and/or a microprocessor, which may include the data processing unit. The control unit and/or the data processing unit is preferably configured to perform a control process, which is also called a control software or a control program. The functions of the incubator and/or of the control unit can be described in method steps. They can be implemented as components of the control program, in particular as subroutines of the control program.

Preferably, the laboratory temperature control device is a laboratory temperature control cabinet, in particular an incubator. The incubator is a laboratory incubator and thus a device with which controlled climate conditions for various biological development and growth processes can be created and maintained. It serves in particular to create and maintain a microclimate with controlled gas and/or humidity and/or temperature conditions in the incubator chamber, whereby this treatment can be time-dependent. The laboratory incubator, in particular a treatment unit of the laboratory incubator, may in particular comprise a timer, in particular a timer, a temperature control unit configured as a heating and/or cooling unit and preferably an adjustment for controlling an exchange gas supplied to the incubator chamber, an adjustment unit for the composition of the gas in the incubator chamber of the incubator, in particular for adjusting the CO₂ and/or the O₂ and/or the N₂ content of the gas and/or an adjustment unit for adjusting the humidity in the incubator chamber of the incubator.

The incubator comprises in particular the incubator chamber (=chamber), furthermore preferably a control unit with at least one control circuit, to which the at least one temperature control unit is assigned as a final control element and at least one temperature sensor as a measuring element. Depending on the embodiment, it can also be used to control the air humidity, although the air humidity itself is not measured by an air humidity sensor (rH sensor) and the air humidity is not the input variable of the control loop. A tub filled with water in the incubator chamber can be heated or cooled to adjust the humidity via evaporation. CO₂-incubators are used in particular for the cultivation of animal or human cells. Incubators may comprise turning devices for turning the at least one cell culture vessel and/or a shaking device for shaking or moving the at least one cell culture vessel.

The control unit can be configured to automatically select a program parameter or an incubator control parameter depending on other data. A treatment, controlled by a control parameter, of the at least one cell culture in at least one cell culture container corresponds in the case of an incubator in particular to a climate treatment to which the at least one cell culture is subjected. Possible parameters, in particular program parameters, in particular user parameters, which are used to influence a climate treatment, define in particular the temperature of the incubator room in which the at least one sample is incubated, the relative gas concentration of O₂—and/or CO₂ and/or N₂ in the incubation interior, the air humidity in the incubation interior and/or at least one sequence parameter which influences or defines the sequence, in particular the order, of an incubation treatment program consisting of several steps.

The temperature control unit can be a combined heating/cooling unit. It is preferably only a heating unit. This can in particular generate the heat via an electrical resistance wire.

The laboratory temperature control device or the incubator can comprise exactly one chamber, but can also comprise several chambers, whose atmosphere (temperature, relative gas concentration, humidity) can be adjusted individually or collectively in particular. A typical size of the interior of a chamber is between 50 and 400 liters, although smaller chamber sizes are also possible for special applications (IVF), in particular 10 to 49 liters.

The features and preferred embodiments mentioned within the scope of the invention of the laboratory temperature control device according to claim 1 can also be used to configure a laboratory temperature control device according to the second or third special aspect. Also the laboratory temperature control device according to claim 1 can be configured by features of the laboratory temperature control device according to the second or third special aspect. Further preferred embodiments of the laboratory temperature control device according to the invention can be found in the description of the embodiments according to the figures.

It shows:

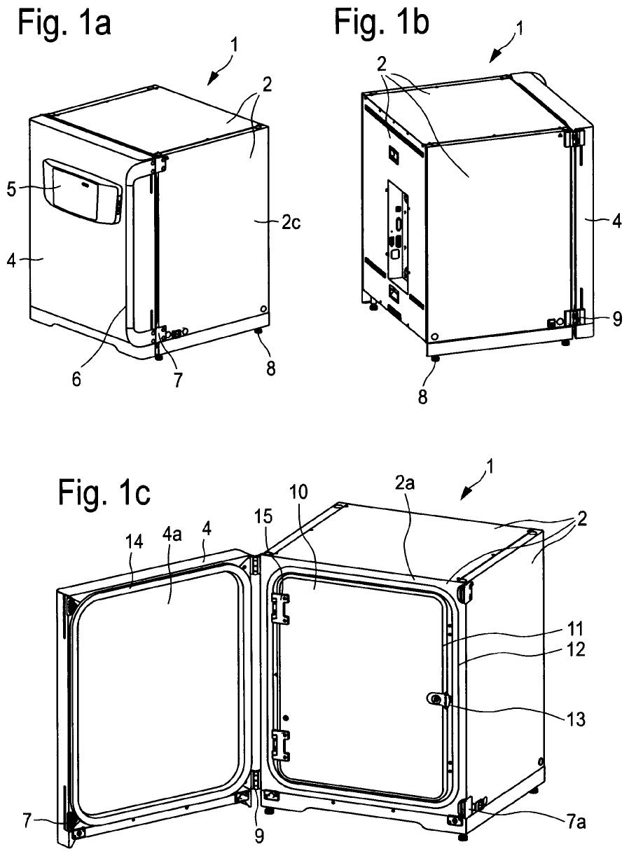

FIG. 1a shows a perspective lateral-frontal view of an incubator according to an embodiment, with the housing door closed.

FIG. 1b shows a perspective side-back view of the incubator of FIG. 1a.

FIG. 1c shows a perspective lateral-frontal view of the incubator of FIG. 1a, when the housing door is open.

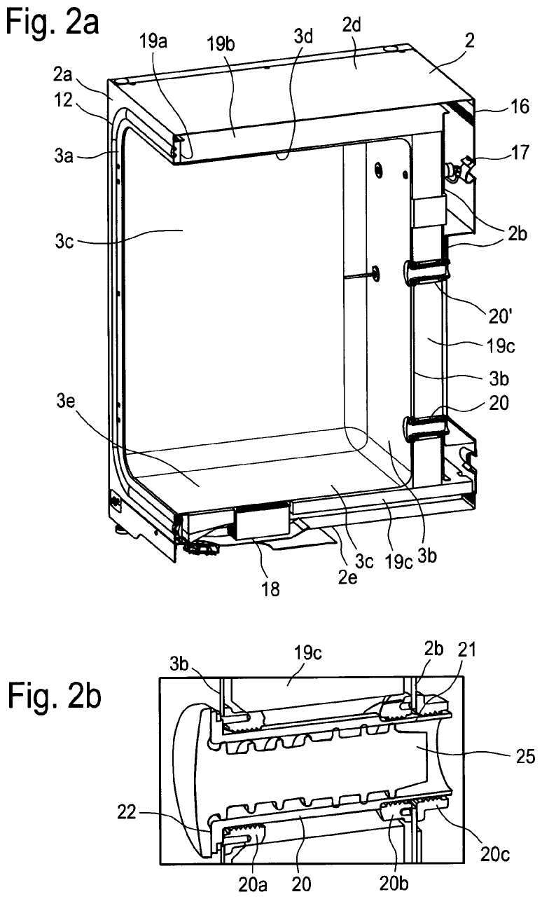

FIG. 2a shows a perspective side-frontal view of the incubator of FIG. 1a, with the housing door hidden and in a cross section along a plane parallel to the side wall and running centrally through the incubator.

FIG. 2b shows as detail of FIG. 2a one of the access ports of the incubator serving as spacer elements between chamber and housing.

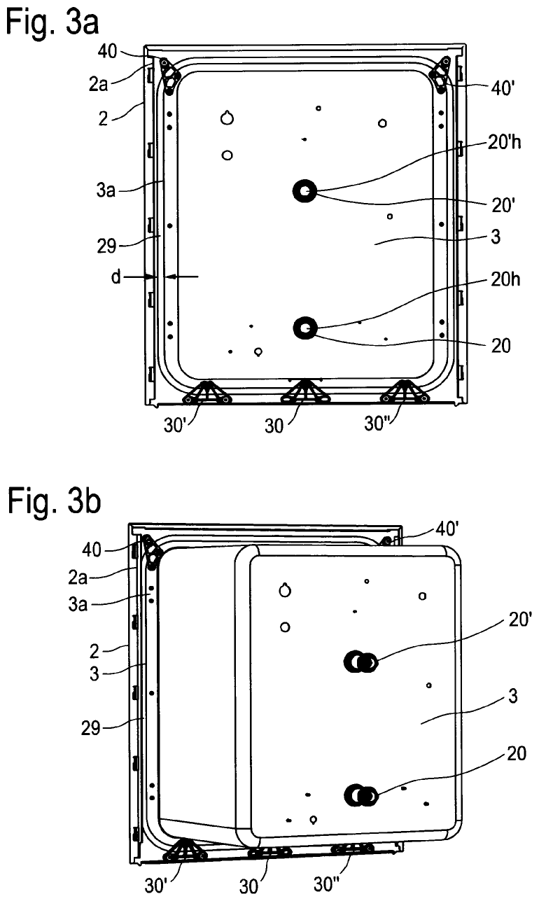

FIG. 3a shows a perspective rear view of the front wall of the housing and the chamber, of the spacer elements arranged between the front wall of the housing and the front panel of the chamber and of the access ports of the incubator of FIG. 1a arranged on the rear wall of the chamber.

FIG. 3b shows a perspective side-rear view of the parts shown in FIG. 3a.

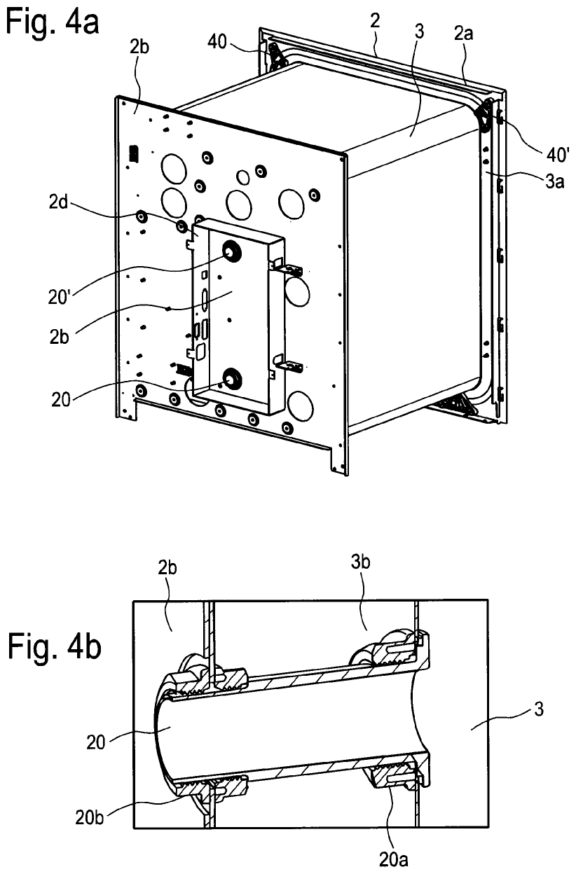

FIG. 4a shows a perspective side-rear view of the parts shown in FIG. 3a as well as a part of the rear wall of the housing with attachments.

FIG. 4b shows a cross-sectional view of an access port serving as a spacer element according to the arrangement in FIG. 4a.

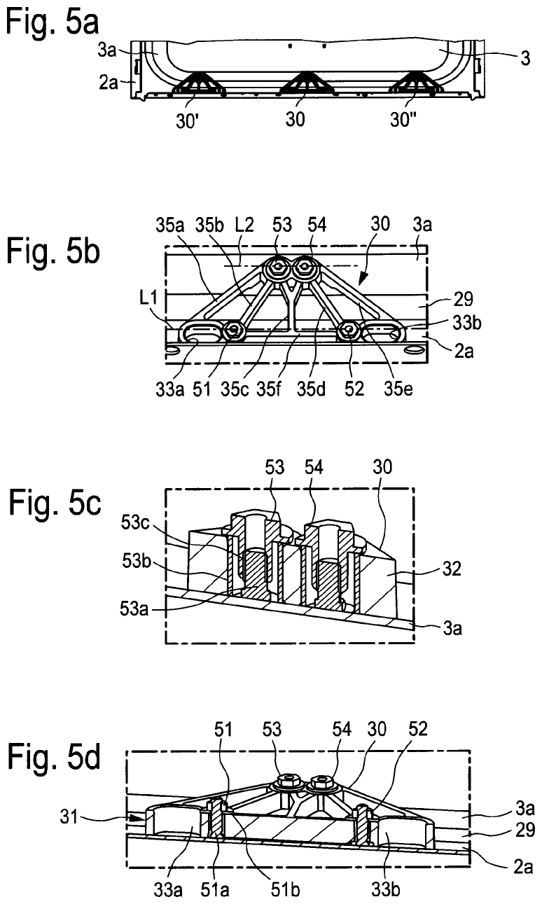

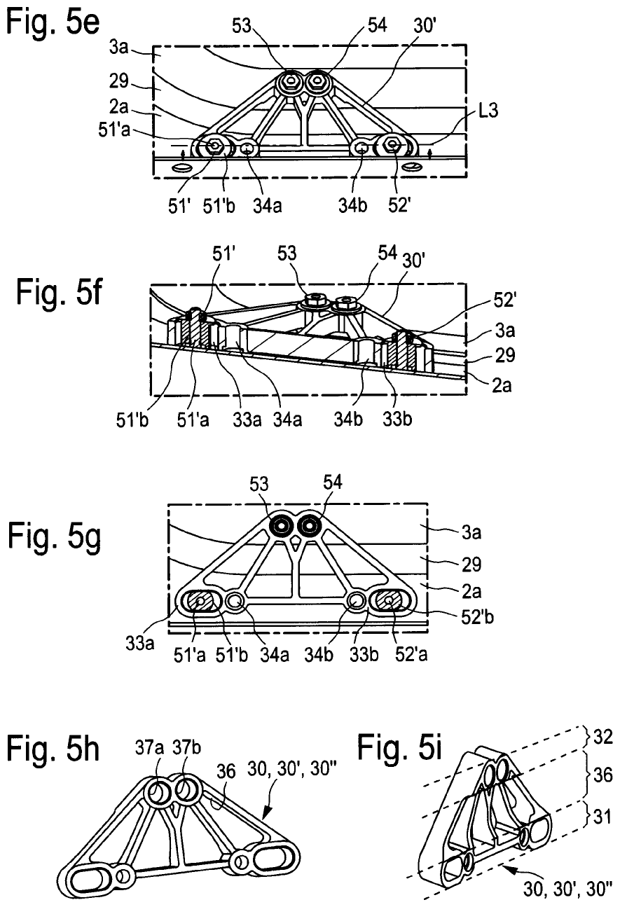

FIG. 5a shows a section of a perspective rear view of the front wall of the housing and the chamber in the lower area of the incubator of FIG. 3a and of the spacer elements arranged there between the front wall of the housing and the chamber front panel.

FIG. 5b shows as a cutout of FIG. 5a the spacer element, which is there centrally located.

FIG. 5c shows a perspective cross-sectional view of the chamber-sided second connecting section of the spacer element of FIG. 5b, wherein the cross-section is perpendicular to the main plane of the plate-shaped spacer element and along the line L2 of FIG. 5b.

FIG. 5d shows a perspective cross-sectional view of the housing-sided first connecting section of the spacer element of FIG. 5b, wherein the cross-section is perpendicular to the main plane of the plate-shaped spacer element and along the line L1 of FIG. 5b.

FIG. 5e shows as a cutout of FIG. 5a the spacer element shown there on the left side of the figure.

FIG. 5f shows a perspective cross-sectional view of the housing-sided first connecting section of the spacer element of FIG. 5e, wherein the cross-section is perpendicular to the main plane of the plate-shaped spacer element and runs along the line L3 of FIG. 5e.

FIG. 5g shows a view of the spacer element of FIG. 5e.

FIG. 5h shows a perspective view of the main side of the also in FIG. 5e shown plate-shaped spacer element.

FIG. 5i shows a perspective view of the main side of the plate-shaped spacer element, which is also turned away in FIG. 5h and which is not visible there and which, when mounted, faces the front wall of the incubator.

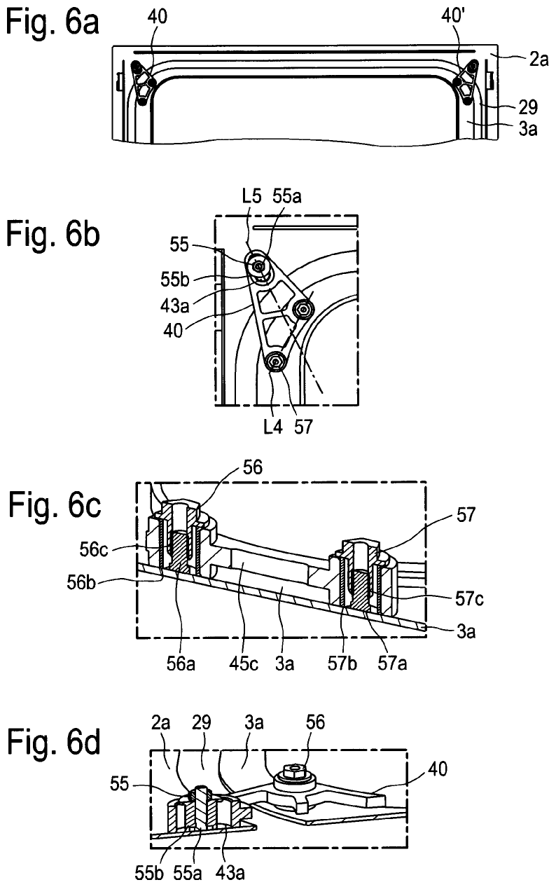

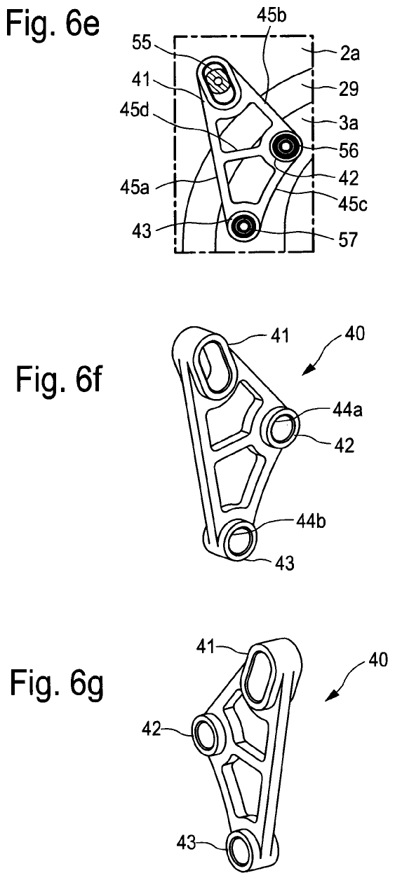

FIG. 6a shows a cutout of a perspective rear view of the housing front wall and of the chamber in the upper area of the incubator of FIG. 3a and the spacer elements arranged there between the housing front wall and the chamber front panel.

FIG. 6b shows as a cutout of FIG. 6a the spacer element visible there on the left side of the figure.

FIG. 6c shows a perspective cross-sectional view of the chamber-sided second connecting sections of the spacer element of FIG. 6b, wherein the cross-section is perpendicular to the main plane of the plate-shaped spacer element and along the line L4 of FIG. 6b.

FIG. 6d shows a perspective cross-sectional view through the housing-sided first connection section of the spacer element of FIG. 6b, wherein the cross-section is perpendicular to the main plane of the plate-shaped spacer element and along the line L5 of FIG. 6b.

FIG. 6e shows a top view of the spacer element of FIG. 6b.

FIG. 6f shows a perspective view of the also in FIG. 6b shown main side of the plate-shaped spacer element.

FIG. 6g shows a perspective view as an oblique view of the main side of the plate-shaped spacer element, which is also turned away in FIG. 6f and which is not visible there and which, when mounted, faces the front wall of the incubator.

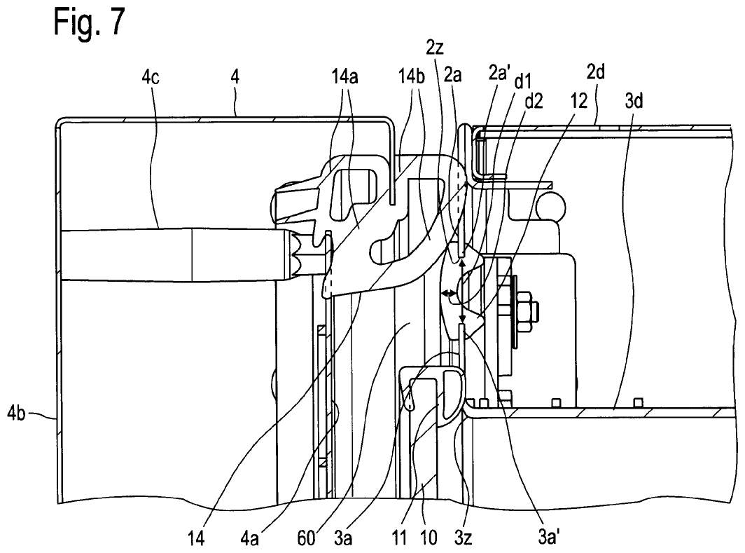

FIG. 7 shows a lateral cross-sectional view of the incubator shown in FIG. 1a, wherein the cross-section runs parallel to a side wall of the incubator and shows an upper front area of the incubator.

FIG. 1a shows the laboratory temperature control device 1 designed as incubator 1 for the growth of cell cultures, here a laboratory temperature control cabinet configured as CO₂ incubator for the growth of eukaryotic cells. The incubator 1 has a housing 2 with an interior surrounded by at least one housing wall 2 and a temperature-controlled chamber 3 (see FIG. 2a), which is located in the housing, with an interior surrounded by at least one chamber wall for holding the laboratory samples. The outer walls of the housing are connected in such a way that they support all other components of the incubator. The housing rests on plinths 8. In normal use, the outer sides of the side walls 2c of the housing, the front wall 2a, the rear wall 2b and the outer side of the housing door 4 and its inner side 4a, as well as the side walls of the chamber, the chamber front wall 3a, and the chamber rear wall 3b are arranged vertically, i.e. parallel to the direction of gravity. The upper outside 2d and the non-visible bottom side of the housing and the bottom and top wall of the chamber are arranged horizontally accordingly. In the context of the description of the invention, the direction “downwards” always refers to the direction of gravity with which a properly operated laboratory temperature control device is aligned; the direction “upwards” is the opposite direction. The “forward” direction indicates the horizontal direction to the front of the closed housing door, the “backward” direction indicates the horizontal direction to the back of the incubator. The chamber is made of stainless steel, the housing is made of painted sheet metal.

The housing door 4 carries a user interface device 5, which here includes a touch-sensitive display that is used by the user to read and enter information. The housing door has two hinges 9, which connect the housing door with the housing 2. By means of a magnetic locking unit 7, which includes an upper and lower housing-sided holding section 7a and an upper and lower housing door-sided holding section 7b, the housing door is held in the closed position.

The housing door comprises a door handle 6, which is connected to the housing door at the positions of the upper and lower housing door-sided holding sections 7b and which extends vertically.

The housing front wall 2a runs vertically and is aligned with the chamber front wall 3a, which also runs vertically, i.e. the forward facing surfaces, and here also the rearward facing surfaces, of the housing front wall 2a and the chamber front wall 3a are essentially in the same plane, see FIG. 7.

As shown in FIG. 1c, an elastic seal 12 is inserted between the housing front wall 2a and the chamber front wall 3a, where it is form-fit held by means of grooves in which the edges of the housing front wall 2a and the chamber front wall 3a engage, see also FIG. 7.

In FIG. 1c the housing door 4 is shown open. The chamber door 10 is attached to the chamber front wall 3a by means of the hinges 15, and is in the shown position held closed by a magnetic hand lock 13, so that the chamber interior is not accessible. Due to the transparency of the chamber door 10, however, the interior is visible to the user in this position. The chamber door is held gas-tight against the chamber front wall by a surrounding elastic seal 11 of the chamber door. The inner side 4a of the housing door comprises a surrounding elastic seal 14 which, when the enclosure door is closed, rests flush against the housing front wall and the surrounding seal 12 there and achieves a gas-tight shielding of the area between chamber door 10 and enclosure door 4a.

As partly visible in FIG. 2a, the incubator comprises two temperature control units which control the temperature of the chamber interior 3, i.e. adjust its temperature by a temperature control. Some of the necessary components 18 are located between the housing bottom wall 2e and the chamber bottom wall 3e. The heating coils of an upper heating circuit (not shown) are thermally coupled and connected to the outside of the chamber ceiling wall 3d and an upper area of the chamber side walls, here about the upper ⅔ along the height of the side walls 3c of the chamber. The heating coils of a lower heating circuit (not shown) are thermally coupled and connected to the outside of the chamber bottom wall 3e and a lower area of the chamber side walls, here about the lower ⅓ along the height of the side walls 3c of the chamber.

A thermal insulation unit 19 is provided between chamber and housing. It isolates the chamber, with adjacent temperature control units, from the housing, which on its outside is in direct contact with the environment. The incubator normally operates at outside temperatures between 18° C. and 28° C. The temperature control units or the temperature control works particularly efficiently in this area. The insulating unit comprises a U-shaped bent insulating element 19b made of glass wool or mineral wool, which surrounds the chamber ceiling plate and the two chamber side walls 3c. It opens to the floor and the rear wall on insulating panels 19c made of PI R foam (polyisocyanurate foam), and is sealed to the front of the housing and chamber by a surrounding needlefelt strip 19a, which lies against the inside of the housing front wall 2a, the chamber front wall 3a and the seal 12. The thermal insulation of the chamber to the outside is optimized by the inventive measures.

A double rear panel 16 is attached to the rear panel 2b to cover rear mounted components. The rear panel is removable by means of a handle 17.

As shown in detail in FIG. 2b, the incubator comprises two access ports 20, 20′ at its rear side, allowing the user to run cables into the inside of the chamber through openings 20h, 20′h in the rear wall of the chamber, e.g. to control measuring instruments placed inside. A port 20, 20′ in this case also serves as a spacer element that keeps chamber 3 at a distance from housing 2 and supports part of the weight of the chamber and its attachments and contents. For this purpose, the access port has a cylindrical component 20 which is made of a material with low thermal conductivity of less than 15 W/(mK), here PPS. This comprises a flange 22, which serves as a second connecting section, which lies against the inside of the chamber back wall 3b and is pressed against the chamber back wall 3b by means of a first threaded ring 20a made of PPS. The outward facing end 21 of the cylindrical component 20 serves as the first connecting section by means of which the spacer element 20 is attached to the housing. For this purpose, a third threaded ring 20b and a second threaded ring 20b are screwed onto the cylindrical component 20 so that the housing rear wall 2b is fixed between the second and third threaded ring. For fastening the threaded rings 20a, 20b, 20c, the cylindrical component 20 comprises corresponding external threads. The spacer element 20′ has an analogus configuration. When the access port is not needed, it is filled by a plug 25 made of thermally insulating material, e.g. silicone foam.

According to the invention, chamber 3 is kept at a distance from the housing by several spacer elements which have a thermal conductivity of less than 15 W/(mK), here approx. 0.5 W/(mK) each, by using a PPS reinforced with fiber fillers. The incubator has several spacer elements 30, 30′, 30″, 40, 40′, 20, 20′, wherein a spacer element has at least one first connecting section 31, 41, 21 by means of which the spacer element and the housing 2 are connected and has at least one second connecting section 32, 42, 22 spaced from the first connecting section by means of which the spacer element and the chamber are connected.

FIG. 3a shows a perspective rear view of the housing front wall 2a and of the chamber 3, of the spacer elements 30, 30′, 30″, 40, 40′ located between the housing front wall 2a and of the chamber front wall 3a and the access ports 20, 20′ of the incubator of FIG. 1a located on the chamber rear wall 3b, which also serve as spacer elements.

By the front spacer elements 30, 30′, 30″, 40, 40′ the chamber front wall 3a, which is aligned with the housing front wall 2a, is kept at a distance d, which is constantly approximately 14 mm. This results in a gap 29 around the chamber opening, which is filled by the thermally insulating seal 12. The spacer elements 30, 30′, 30″, 40, 40′ are designed according to the invention in such a way that, on the one hand, they can easily and reliably support the main part of the weight of the chamber, as well as its attachments and its maximum permissible filling weight over the entire life of the incubator. The number of front spacer elements results from the intersection of the requirements for the mechanical, chemical and thermal load-bearing capacity of the connection and the thermal insulation capacity. These parameters can be influenced on the one hand by the suitable material selection and on the other hand by the advantageous geometric design of the spacer elements.

As material of the spacer elements, from which they were integrally manufactured in particular by an injection moulding process, a high-performance plastic was chosen here, in particular a composite material with a matrix of high-performance plastic. This was chosen in this case as PPS GF 40, i.e. a PPS with 40% addition of glass fibers, which was here provided with a further 25% addition of mineral fillers. This results in an excellent thermal load capacity of up to 220° C. This allows the chamber to be easily heated up to 180° C. for sterilization purposes, which is a standard requirement for modern incubators. The thermal expansion of the said PPS material at 20° C., measured in particular between 20° C. and 60° C., is 15*10-6 K-1 in the longitudinal direction to the glass fiber, and 30*10-6 K-1 in the transverse direction to the glass fiber. The thermal conductivity of the PPS material is only 0.5 W/(mK), resulting in a high thermal resistance, ideal for thermal decoupling of chamber and housing. The tensile strength of the PPS material according to ISO 527 is approximately 150 MPa.

The geometric structure of the front spacer elements was optimized with regard to the load to be carried and with regard to reduce the heat flow. For this purpose, in particular the cross section determining the heat flow was minimized by the “third section” located between the first and second connecting section of the spacer element 30. On the other hand, the distance to be covered by the heat flow was aimed at by maximizing the length of the third section. For this purpose, the floor-side spacer elements 30, 30′, 30″ were constructed in such a way that they comprise several web-shaped sections 35a, 35b, 35c, 35d, 35e—each of identical construction—which connect the first connecting section 31 with the second connecting section 32.

A front-sided spacer element 30, 30′, 30″, 40, 40′ is at present a substantially plate-shaped component comprising two opposite main sides, namely the rear side of the spacer visible in FIG. 5h and FIG. 6f and the front side of the spacer visible in FIG. 5i and FIG. 6g, which extend parallel to the main plane of the plate-shaped component. The views in FIGS. 5g and 6e are parallel to this main plane. The cross-section of the front-sided plate-shaped spacer elements parallel to its main plane essentially follows a triangular contour, in the case of the bottom-sided spacer elements according to an isosceles triangle, resulting in a favorable distribution of forces. The chamber loads the bottom-sided spacer elements 30, 30′, 30″ with a pressure load, the top-sided spacer elements are loaded with a tensile load, the chamber is here suspended on the upper side of the housing front wall.

The first connecting section 31 of the bottom-sided spacer element 30 is here a beam-shaped area 31, see FIG. 5i, which contacts the front wall of the housing 2a in the assembled state and is supported against the front wall of the housing 2a by fasteners 51, 52, 51′, 52′. The first connecting section 31 comprises two sections with cylindrical holes 34a and 34b spaced apart and connected by the web-shaped connecting section 35f, the cylindrical axes of which extend perpendicular to the main plane of the plate-shaped spacer element 30. The holes 34a and 34b are used to mount the central spacer element 30 as shown in FIGS. 5b to 5d.

As shown in FIG. 5b, a spacer element 30, 30′, 30″ is formed by a plate-shaped component with approximately the contour of an isosceles triangle. The outer ridge-shaped sections 35a, 35e form an acute angle with the horizontal and at the bottom of the triangle arranged ridge-shaped section 35f, in particular the same acute angle, which is about 41° here. At the meeting point of the web-shaped sections 35a, 35e with the web-shaped section 35f, the oval-cylindrical section with oblong hole 33a, 33b and the cylindrical section with cylindrical hole 34a, 34b are respectively located adjacent to each other. The outer web-shaped sections 35a, 35e each start from the oval-cylindrical section and end at the apex of the isosceles triangle at an obtuse angle, wherein the apex is represented by the first connecting section 31, which is formed by two adjacent cylindrical sections each with a cylindrical hole 37a, 37b. Two further web-shaped sections 35b, 35d enclose an angle of approx. 57° with the web-shaped section 35f. In the center of the spacer element, the web-shaped section 35c extends vertically upwards, perpendicular to the web-shaped section 35f and ends in the apex of the triangle by bifurcating in a Y-shape, with each bifurcation ending in one of the cylindrical sections of the apex. Due to this strut construction, the spacer element 30 has a high mechanical stability, especially against the pressure load from above, a low weight, and a high thermal resistance against the heat flow, which results from the first connection section 31 through the struts 35a, 35b, 35c, 35d, 35e to the second connection section 32. The above-mentioned struts have a relatively long length and a small cross section seen transversely to the heat flow, which minimizes the heat flow.

As can be clearly seen in FIG. 5i, the spacer element 30 comprises a recess 36 on its front side, which is shaped to accommodate another component, here to partially accommodate the seal 12, which is arranged in the gap 29. The recess further reduces the cross-section mentioned in the above paragraph and increases the thermal resistance. The web-shaped struts 35a, 35b, 35c, 35d, 35e running in the area of the recess 36 are each also regarded as a third section of the spacer element, which connects a first and second connecting section each.

FIG. 5d shows a perspective cross-sectional view of the housing-sided first connection section 31 of the centrally located spacer element of FIG. 5b, wherein the cross-section is perpendicular to the main plane of the plate-shaped spacer element and along the line L1 of FIG. 5b. Two externally threaded metal pins 51a are welded to the back of the front wall of the housing 2a so that they each extend into a hole 34a, 34b of the spacer element. A washer 51b and the nut 51 screwed onto the metal pin 51a are used at each hole 34a, 34b to press the first connection section 31 against the front wall 2a of the housing to make the connection.

The second connecting section 32 of the floor-sided spacer element 30, 30′, 30″ is formed by a narrower beam-shaped section 32, see FIG. 5i, which contacts the chamber front wall 3a in the assembled state and is supported by fasteners 53, 54 against the chamber front wall 2a. The second connecting section 32 comprises two cylindrical holes 37a, 37b, see FIG. 5h, whose cylinder axes extend perpendicular to the main plane of the plate-shaped spacer element 30. The holes 37a and 37b are used to mount the spacers 30, 30′, 30″ on the chamber front wall 3a, as shown in FIG. 5c. In each of the holes 37a and 37b a precisely fitting cylindrical metal sleeve 53b is mounted, the length of which corresponds to the height of the spacer element in this direction. The metal pins 53a, which have a external thread, and which are welded to the chamber front wall 3a project into the metal sleeve. A nut head 53, 54 is screwed to the metal pin 53a using thread 53c and presses against the metal sleeve and spacer element 30, which is therefore relieved by the metal sleeve.

FIG. 5e shows as an cutout of FIG. 5a the distance element 30′ shown there on the left side of the figure. In the case of the decentralized, i.e. laterally arranged bottom-sided spacer elements 30′ and 30″, the oblong holes provided on the first connecting section are used to fasten the spacer element 30′, 30″ to the front wall of the housing while tolerating a horizontal sliding movement. The spacer elements 40, 40′ on the upper side of the chamber also have such a sliding fastening by means of a slotted hole. A “floating attachment” of the chamber to the housing is achieved by means of the slotted holes. This type of bearing arrangement makes it possible to compensate for thermally induced length changes in the housing or to reduce the mechanical stresses caused by the thermally induced length changes. Only the spacer element 30, which is centrally located at the bottom, is not slidingly fixed and guarantees the positioning of the chamber even in the presence of thermally induced length changes.

FIG. 5f shows a perspective cross-sectional view of the housing-sided first connecting section of the decentrally laterally arranged spacer element 30′ of FIG. 5e, wherein the cross-section is perpendicular to the main plane of the plate-shaped spacer element and along the line L3 of FIG. 5e. Two metal pins 51′a are each welded to the housing front wall 2a and protrude through the oblong hole 33a, 33b. A cylindrical sliding sleeve 51′b, 52′b surrounds the metal pin 51′a, 52′a and ends in a disc-shaped sleeve head which is pressurized by the nut 51′, 52′ which is screwed onto the thread of the metal pin 51′a, 52′a.

FIG. 6b shows as a cutout of FIG. 6a the distance element 40 visible there on the left side of the figure. It comprises a first connecting section 41, which, as shown in FIG. 6f and FIG. 6g, is shaped as an oval-cylindrical section 41, through which slot 43a extends. This section 41 is mounted in the assembled position against the front wall of the housing 2a. For this purpose, it comprises a metal pin 55a welded to it, see FIG. 6d, which is surrounded by a sliding sleeve 55b, which is fitted into the oblong hole 43a in order to carry out possible mechanical stresses there by a sliding movement of the housing section with the metal pin 55a relative to the spacer element 41 connected to the chamber front wall 3a. The nut 55 is screwed to the metal pin 56a and presses the metal sleeve against the housing front wall 2a in such a way that a sliding movement of the distance element 40 between the housing front wall 2a and the metal sleeve 55b is enabled.

FIG. 6c shows a perspective cross-sectional view of the chamber-sided second connecting sections 42, 43 of the spacer element 40 of FIG. 6b, wherein the cross-section is perpendicular to the main plane of the plate-shaped spacer element and along the line L4 of FIG. 6b. The sections 42, 43 called “second connecting sections” connect the spacer element 40 with the chamber front wall 3a. The connecting sections 42 and 43 are cylindrical sections of the spacer element 40, as shown in FIG. 6f, 6g, through each of which a cylindrical hole 44a, 44b extends. These serve to fasten the spacer element 40 to the chamber front wall 3a. For this purpose it comprises two metal pins 56a, 57a welded to it. Each of these pins extends into a hole 44a, 44b of the distance element 40. Each hole is fitted with a metal sleeve 56b, 57b, which encloses the hole. By means of a nut 56, 57, which respectively extends into the hole, which rests on the metal sleeve and which is connected to the external thread 56c, 57c of the metal pin 56a, 57a, each of the connecting sections 42, 43 between the metal sleeve 56b, 57b and the chamber front wall 3a and is mounted and fastened in such a way that the compressive force is absorbed by the metal sleeve and the spacer element is relieved.

FIG. 6e shows a view of the spacer element of FIG. 6b, in which the web-shaped sections 45a, 45b, 45c, 45d between the connecting sections 41, 42, 43 are marked. The web 45a connects the connecting section 41 with the connecting section 43, the web 45b connects the connecting section 41 with the connecting section 42, the web 45c connects the connecting section 42 with the connecting section 43, web 45d connects the connecting section 42 with the web 45a. In this way, a lightweight, mechanically strong construction is created, whose thermal resistance is maximized due to the small cross section of the webs 45a, 45b perpendicular to the heat flow direction between the first and second connecting sections and the relatively long length of these webs. As can be clearly seen in FIG. 6c, the web 45c—unlike the web 35f of the spacer element 30 (FIG. 5f)—is not in contact with the corresponding front wall panel: in FIG. 6c the web is separated from the chamber front wall 3a by a gap. The sections 42 and 43 of the spacer element are each individually supported on the chamber front wall and therefore, unlike the beam-shaped section 31 in FIG. 5f, are considered as two different connecting sections.

The spacer element 40′ located in the other upper corner of the chamber front wall 3a is formed analogous to the spacer element 40, but is mirror-inverted to it, and is connected in the same way to the chamber front wall 3a and to the housing front wall 2a.

FIG. 7 shows a lateral cross-sectional view of the incubator shown in FIG. 1a, wherein the cross-section runs parallel to a side wall of the incubator and shows an upper front area of the incubator. There an upper area of the enclosure door 4 is shown in sections, in the closed position of the outer door 4, in which this upper area of the outer door abuts the enclosure front wall 2a by means of seals. Chamber 3 comprises a chamber door 10, which is also closed here. The housing front wall 2a and the chamber front wall 3a are separated from each other by the first seal 12, which surrounds the chamber opening. The first seal is made of silicone foam. The outer door 4 comprises an inner side 4a with a second seal 14 which, when the outer door is closed, rests against the first seal 12 and surrounds the chamber opening 3z and the housing opening 2z.

Such a laboratory temperature control device according to the invention offers the advantage that the space 60 formed between the chamber door and the housing door when the outer door is closed is not in contact with the housing. This is due to the fact that, when the outer door is closed, the second seal laterally limits the space between the two seals by ensuring uninterrupted contact with the first seal. Any contact between the air mixture heated by the chamber and the non-tempered outer wall of the housing and thus any convection-induced heat transfer between the interstitial space and the housing is therefore impossible.

The first seal closes the gap 29 between the housing front wall 2a and the chamber front wall 3a. The gap width d here is d1=14 mm. The width of the first seal 12 in the direction of d1 is slightly more than 14 mm, since the planar sheet metal ends of the housing front wall 2a and the chamber front wall 3a each engage from opposite sides in a corresponding groove in the first seal, resulting in a form-fit connection between the first seal and the housing front wall 2a and the chamber front wall 3a. The minimum thickness of the first seal here is d2=3.0 mm. Due to the mentioned preferred embodiments (maximum heat flow distance in the direction of the width of the gap and minimum heat conducting cross-section perpendicular to this direction) the thermal resistance of the first seal 12 is maximum.

The second seal has a circumferential first sealing area 14a which is located closer to the outer door and which has a higher modulus of elasticity than the second sealing area 14b, which is integrally connected to the first sealing area, which is in particular softer and which, in the closed position of the outer door, is closer to and contacts the first seal. The softer sealing area 14b between the outer door 4 and housing 2 provides better sealing of the space between the outer door and the chamber door and also reduces the forces required to close the chamber door, making it easier for the user to operate. The first sealing area is manufactured using silicone, the second sealing area 14b is manufactured using a silicone foam.

Due to the closed space between outer door 4 and chamber door 10, which is not in contact with the relatively cool front wall of the housing, the heat flow through the front area of the incubator is reduced and the thermal decoupling between chamber and outside world is further improved.

The outer door comprises a heated inner wall 4a. This is held, without directly touching the outer wall 4b of the outer door, by several spacer elements 4c on the outer wall 4b of the outer door. A spacer element 4c is preferably made of a material with a thermal conductivity of less than 15 W(mK), in particular made of a high performance plastic, in particular PPS. This further improves the thermal decoupling of the chamber from the environment.

The wall end section 2a′ of the housing front wall surrounding the chamber front wall 2a forms the housing opening 2z, and the chamber front wall 3a forms a flange around the chamber opening 3z, which ends in a wall end section 3a′ surrounding the chamber opening. The wall end section 2a′ of the housing front wall and the wall end section 3a′ of the chamber front wall each have a thickness of approx. 2.0 mm and face each other to form the gap 29. The already thin wall end sections are connected to each other by the first seal 12. The wall end section 2a′ of the housing front wall and the wall end section 3a′ of the chamber front wall are presently also in the same plane. The heat input of the wall end section 3a′ of the chamber front wall into the seal is low due to the small wall thickness, so that the heat transfer is further reduced. This configuration minimizes the heat transfer between wall end section 2a′ of the enclosure front wall and wall end section 3a′ of the chamber front wall by means of thermal radiation and further improves the thermal decoupling between chamber and enclosure.

Through the measures of thermal decoupling of chamber and housing described here, it was surprisingly possible to reduce the energy consumption of the incubator according to the invention by about 50% compared to an incubator of the current generation, which illustrates the efficiency of the mentioned measures.

### LIST OF REFERENCE SINGS

1: Laboratory temperature control cabinet, incubator
2: Housing, housing wall
2a: Front wall of the housing
2a′: Wall end section of the housing front wall
2b: Rear wall of the housing
2c: Side walls of the housing
2d: upper outside of the housing
2e: Housing bottom wall
2z: Housing opening
3: Chamber/chamber interior
3a: chamber front wall
3a′: Wall end section of the chamber front wall
3b: Chamber rear wall
3c: Side wall of the chamber
3d: Ceiling wall of the chamber
3e: Chamber bottom wall
3z: Chamber opening
4: Outer side of the housing door/outer door
4a: Heated inner side of the housing door/outer door
4b: Outer wall of housing door/outer door
4c: Spacer element
5: User interface device
6: Handle outer door
7: Locking unit
7a: Housing-sided holding section of the locking unit
7b: Housing door-sided holding section of the locking device
8: Plinth
9: Hinge
10: Chamber door; transparent
11: Elastic seal
12: Elastic seal; first seal
13: Magnetic manual closure
14: Elastic seal; second seal
14a: First sealing area made of unfoamed silicone
14b: Softer sealing area made of silicone foam; second sealing area
15: Hinge
16: Double outer rear wall
17: Handle of the rear wall
18: Components for temperature control
19: Insulating unit
19a: Needle fleece strip of the insulating unit; surrounds the chamber
19b: U-shaped bent insulating element of insulating unit 19 made of glass wool
19c: Insulating plate of the insulating unit 19 made of PIR foam
20: Access port
201: Access port; spacer element
20h: Opening of the access port in the rear wall of the chamber
20′h: Opening of the access port in the chamber rear wall
20a: Threaded ring
20b: Threaded ring
20c: Threaded ring
21: Outer end of the access port; first connection section
22: Access port flange; second connection section
25: Plug of the access port
29: Gap
30: Spacer element
301: Spacer element
30″: Spacer element
31: First connection section
32: Second connection section
33a: Elongate hole
33b: Elongate hole
34a: Cylindrical holes
34b: Cylindrical holes
35a: Web-shaped section
35b: Web-shaped section
35c: Web-shaped section
35d: Web-shaped section
35e: Web-shaped section
35f: Web-shaped section
36: Recess
37a: Cylindrical hole
37b: Cylindrical hole
40: Spacer element
401: Spacer element
41: First connection section
42: Second connection section
43: Third connection section
43a: Elongate hole
44a: Cylindrical hole
44b: Cylindrical hole
45a: Web-shaped section; bar; webs
45b: Web-shaped section
45c: Web-shaped section
45d: Web-shaped section
51: Fastener; nut
51′: Fastener; nut
51a: Metal pin
51a′: Metal pin
51b: Washer
51′b: Cylindrical sliding sleeve
52: Fasteners
52′: Fastener; nut
52a′: Metal pin
52′b: Cylindrical sliding sleeve
53: Fastener; nut head
53a: Metal pin
53b: Metal sleeve
53c: Thread
54: Nut head
55: Nut
55a: Metal pin
55b: Sliding sleeve; metal sleeve
56: Nut
56a: Metal pin
56b: Metal sleeve
56c: Thread
57: Nut
57a: Metal pin
57b: Metal sleeve
57c: Thread
60: Space between chamber door and housing door

1. Laboratory temperature control device (1), in particular incubator for cell cultures, for temperature-controlled storage of laboratory samples, comprising
a housing (2) with a housing interior surrounded by at least one housing wall,
a temperature-controlled chamber (3) arranged in the housing with a chamber interior surrounded by at least one chamber wall for receiving the laboratory samples,
a plurality of spacer elements (30; 30; 30″; 40; 40′; 20; 20′), wherein a spacer element comprises at least one first connecting portion (31; 41; 21) by means of which the spacer element and the housing are connected and comprises at least one second connecting portion (32; 42; 22) spaced from the first connecting portion by means of which the spacer element and the chamber are connected so that the chamber is held spaced from the housing by means of the spacer elements,
wherein the spacer elements (30; 30′; 30″; 40; 40; 20; 20′) are each formed using a material having a thermal conductivity of less than 15 W/(mK).

2. Laboratory temperature control device according to claim 1, wherein at least one of the spacer elements is manufactured using a high-performance plastic which tolerates operating temperatures of 180° C.

3. Laboratory temperature control device according to claim 2, wherein the high-performance plastic is polyphenylene sulfide (PPS) or polyetheretherketone (PEEK).

4. Laboratory tempering device according to claim 2, wherein the high-performance plastic comprises fiber additives, in particular glass fiber, for mechanical reinforcement.

5. Laboratory temperature control device according to claim 1, wherein at least one spacer element is a plate-shaped component.

6. Laboratory temperature control device according to claim 1, wherein at least one spacer element comprises at least one recess and/or at least one cavity.

7. Laboratory temperature control device according to claim 1, wherein at least one spacer element comprises a plurality of web-shaped sections.

8. Laboratory temperature control device according to claim 7, wherein at least one web-shaped section connects the first connecting portion and the second connecting portion and/or wherein at least one web-shaped section connects two first connecting portions or two second connecting portions.

9. Laboratory temperature control device according to claim 1, wherein
the housing comprises a housing front wall (2a) and the chamber comprises a chamber front wall (3a), wherein the housing front wall and the chamber front wall are separated from each other by a first seal (12) which surrounds the chamber opening (3z),
wherein an outer door of the housing comprises an inner side with a second seal (14) which, when the outer door is closed, abuts the first seal and surrounds the chamber opening.

10. Laboratory temperature control device, in particular incubator for cell cultures, comprising
a housing with an outer door and a housing opening which can be closed by the outer door and which is formed in a housing front wall
a chamber (3) arranged in the housing and surrounding a chamber interior with a chamber opening (3z) formed in a chamber front wall (3a)
wherein the housing front wall (2a) and the chamber front wall (3a) are separated from each other by a resilient first seal (12) surrounding the chamber opening,
wherein the outer door (4) comprises an inner side (4) with a second seal (14) which, in the closed state of the outer door, abuts the first seal (12) and surrounds the chamber opening (3z).

11. Laboratory temperature control device according to claim 10, wherein the first seal comprises a minimum material thickness (d2) which, measured perpendicular to the front wall of the housing, is less than the gap width (d1) of the gap between the housing front wall and the chamber front wall.

12. Laboratory temperature control device according to claim 10, wherein the second seal in the closed position of the outer door also abuts the front wall of the housing (2a).

13. A laboratory temperature control device according to claim 10, wherein the second seal (14) separates a heated inner side (4a) of the outer door (4) from the outer wall of the outer door.

14. Laboratory temperature control device according to claim 10, comprising a plurality of spacer elements, wherein a spacer element comprises at least a first connecting portion by means of which the spacer element and the housing are connected and at least a second connecting portion spaced from the first connecting portion by means of which the spacer element and the chamber are connected so that the chamber is held spaced from the housing by means of the spacer elements, wherein the spacer elements are each formed using a material having a thermal conductivity of less than 15 W/(mK).

15. Laboratory temperature control device according to claim 1, which is an incubator (1), in particular a CO₂ incubator for cell cultures.
